(19) United States
(12) Patent Application Publication
(10) Pub. No.: US 2025/0050001 A1
(43) Pub. Date: Feb. 13, 2025

(54) BLOOD TREATMENT MACHINE FOR EXTRACORPOREAL BLOOD TREATMENT AND SYSTEM FOR RECOGNIZING BLOOD OR BLOOD CONSTITUENTS IN A TUBING LINE

(71) Applicant: Fresenius Medical Care Deutschland GmbH, Bad Homburg (DE)

(72) Inventors: Franz-Wilhelm Koerdt, Bad Homburg (DE); Nicolas Beisser, Bad Homburg (DE)

(21) Appl. No.: 18/718,627

(22) PCT Filed: Dec. 12, 2022
(86) PCT No.: PCT/EP2022/085323

§ 371 Date: Sep. 17, 2024

(30) Foreign Application Priority Data
Dec. 13, 2021 (DE) 102021132841.1

(51) Int. Cl. A61M 1/16

(52) U.S. Cl. CPC A61M 1/1692; A61M 2205/3306

(57) ABSTRACT

The present disclosure is directed to a medical system for optically detecting at least one blood constituent in a tubing line received in a tubing receptacle apparatus at a blood treatment machine for extracorporeal blood treatment and a corresponding blood treatment machine, the tubing receptacle apparatus having no electrical or electronic components and no electrically conductive shielding in some embodiments. Light guides are used to transmit light into the tubing receptacle apparatus and from the tubing receptacle apparatus.

### CROSS-REFERENCE TO RELATED APPLICATIONS

The present application is the national stage entry of International Application No. PCT/EP2022/085323, filed on Dec. 12, 2022, which claims priority to Application No. DE 10 2021 132 841, filed in the Federal Republic of Germany on Dec. 13, 2021, the entire contents of which are hereby incorporated by reference.

### TECHNICAL FIELD

The present disclosure relates to the optical detection of blood or blood constituents in a dialysate line or a filtrate line at a blood treatment machine for extracorporeal blood treatment, for the purposes of recognizing a threatening hazardous situation for the patient should, in the case of a fault, for example on account of a rupture of the semipermeable membrane of the dialyser or blood filter or if haemolysis occurs in the blood of the patient in the extracorporeal blood circuit, blood or blood constituents pass through the semipermeable membrane of a dialyser or blood filter to the dialysate side or filtrate side such that there is an accidental loss to the patient of blood or blood constituents together with the dialysate or filtrate to be discarded.

### BACKGROUND

Systems for recognizing blood or blood constituents in a dialysate line or a filtrate line at a blood treatment machine for extracorporeal blood treatment are also referred to as blood leakage detectors (BLDs) and represent a safety apparatus for protecting the patient against an unrecognized, accidental loss of blood or blood constituents together with the dialysate or filtrate to be discarded. They recognize blood or blood constituents in the dialysate or filtrate to be discarded and are thereby distinguished from systems for recognizing an accidental blood loss from a leaky extracorporeal blood circuit outside of the dialyser or blood filter or directly from the vessel access of the patient into the surroundings, for example if there is an accidental dislocation or disconnection of a cannula or a catheter or a tubing connection. In contrast to the blood losses from a leaky extracorporeal blood circuit outside of the dialyser or blood filter, a creeping blood loss by way of the semipermeable membrane of the dialyser or blood filter can hardly be recognized and cannot be recognized quickly enough by the naked eye because the blood or the blood constituents are very diluted in the dialysate or filtrate. Such a blood loss via the semipermeable membrane per unit time depends decisively on the flow rate of the dialysate or filtrate and on the concentration of the blood or blood constituents therein. Therefore, a sufficiently high sensitivity is desirable for such a blood leakage detector. The higher the sensitivity, the earlier a loss of blood can be recognized and the less blood the patient loses in the case of a fault.

Some monitoring apparatuses for recognizing an ingress of blood or haemolysis that are used in the extracorporeal blood treatment are based on the spectroscopic evaluation of the red and green spectrum of light, for example. According to the prior art, blood is recognized in the fluid system by using an optical measurement method, in which the reduction in the intensity of the light on account of the clouding of the dialysis fluid by blood or blood constituents is evaluated. When blood enters the dialysis fluid there is a change in the intensity of the light emerging from the dialysis fluid, which is referred to as clouding here, the change in intensity depending on the wavelength of the light. Blood entering the fluid system can be reliably recognized using the known methods. In addition to blood entering the fluid system as a consequence of a defect of the dialyser or filter, for example on account of a membrane rupture or a detachment of a sealing compound, free haemoglobin may enter the dialysis fluid during an extracorporeal blood treatment in the case of haemolysis of the blood. Haemolysis denotes the lysis (rupturing) of the erythrocytes (red blood cells) in the blood. The erythrocytes essentially consist of the oxygen-binding protein haemoglobin, which gives the erythrocytes, and hence also the blood, the red colour. The haemoglobin is released when haemolysis occurs. During an extracorporeal blood treatment, haemolysis may occur on account of, for example, a shearing flow, for example if the blood is mechanically loaded within a blood-guiding tubing line. Such shearing flows occur, inter alia, if a blood-guiding tubing line of the tubing line system of the blood treatment apparatus is kinked. However, haemolysis may also be caused systemically by the patient. During the extracorporeal blood treatment, haemoglobin may pass through the semipermeable membrane, from the blood side of the dialyser to the dialysate side or filtrate side. Therefore, the haemoglobin can be detected in the blood using the optical measurement methods known from the prior art.

### SUMMARY

A disposable tubing line is understood to mean a tubing line system configured as a disposable or single use or discardable article, or a tubing line which is disposed of or discarded after a single use. In the case of special blood treatment machines for continuous extracorporeal blood treatment (CRRT) or in the case of blood treatment machines for acute extracorporeal blood treatment in intensive-care medicine or in intensive care units (ICUs), the dialysate line or filtrate line is formed as a disposable tubing line, just like the extracorporeal blood circuit, and the necessary medical solutions such as dialysate and substitution fluid or substituate are provided as ready-to-use solutions in solution bags. Such disposable tubing lines, such as dialysate line or filtrate line, are connected from the outside to the functional interfaces of the dialysis machine by the user before an extracorporeal blood treatment is carried out. In the process, inter alia, a tubing portion of the dialysate line or filtrate line is inserted into a tubing receptacle region of a blood leakage detector housing that is arranged on the outside surface of the blood treatment machines and is accessible from the outside. The blood leakage detector housing contains at least a light source, an optical sensor system and measurement electronics. The measurement signal is guided from the blood leakage detector housing to the interior of the blood treatment machines and their closed-loop and open-loop control apparatus by means of electrical lines.

Electromagnetic compatibility (EMC) denotes the characteristic of technical equipment to not interfere with other equipment by way of electric or electromagnetic effects, and to not be interfered with by other equipment (interference immunity). The freely accessible arrangement of the blood leakage detector housing on the outside surface of the blood treatment machines is necessarily accompanied by the known blood leakage detectors being exposed to the ambient electromagnetic radiation, and so the blood leakage detector housing generally has its own electrically conductive shielding against electromagnetic radiation. By way of example, the multiFiltratePRO acute dialysis machine by the applicant, Fresenius Medical Care Deutschland GmbH, is known as such a blood leakage detector. If the terms “interference susceptibility” or “interference immunity” are used in the present case, this means interference by electromagnetic radiation.

Aspects of the present disclosure may help reduce the interference susceptibility of the blood leakage detector, in particular that of the phototransistors which are particularly susceptible to interference, in relation to electromagnetic radiation from the surroundings of the dialysis machine.

Aspects of the present disclosure may also render costly, complex-to-install electrically conductive sheet metal shielding of the blood leakage detector housing, including the connection of the electrically conductive sheet metal shielding to the grounding conductor, entirely dispensable, without simultaneously increasing the interference susceptibility of the blood leakage detector in relation to electromagnetic radiation from the surroundings of the dialysis machine.

Some embodiments described herein may increase the sensitivity of the blood leakage detector, in particular such that a computational blood loss of 0.35 ml/min, or even less, can be reliably detected over the entire range of the usual flow rates of dialysate or filtrate or effluent.

Aspects of the present disclosure may reduce the production costs for the blood leakage detector.

Aspects of the present disclosure may reduce the maintenance and repair costs for the blood leakage detector, in particular by way of increasing the reliability and service life, and by way of a structure that is suitable for maintenance and repair.

Aspects of the present disclosure may provide an alternative blood leakage detector without altering the standard measurement and evaluation method, in particular without software modifications to the measurement and evaluation algorithm.

Aspects of the present disclosure may provide an alternative blood leakage detector without altering the standard handling of the known blood leakage detector during practical operation, in particular without the users needing separate training and in particular without needing to modify the instructions for the user.

Some of the embodiments described herein may involve counteracting an ageing of the green LED which generally needs to be operated under great wear-and-tear at its maximally specified electric current to be able to provide the appropriate significant luminous intensity.

Aspects of the present disclosure may provide a simple and easy-to-repair structure of the blood leakage detector, simplify the assembly of the blood leakage detector and thereby save assembly costs, despite the constricted space in the blood leakage detector housing and the constricted space at the front of the dialysis machine.

The present disclosure relates to a medical system for optically detecting at least one blood constituent in a tubing line at a blood treatment machine for extracorporeal blood treatment. Moreover, the present disclosure relates to a blood treatment machine for extracorporeal blood treatment.

One innovative aspect of the present disclosure relates to a blood treatment machine for extracorporeal blood treatment with a machine housing.

The machine housing can at least hold an electronic open-loop and closed-loop control apparatus in its interior, for open-loop and closed-loop control of extracorporeal blood treatment. Optionally, the machine housing additionally holds at least one or more electric drive motors for driving at least one pump from the group of blood pump, dialysis fluid pump, dialysate pump, filtrate pump, effluent pump in its interior, in particular on the inner side of an outer wall. In some embodiments, the machine housing includes a foot with four rollers so that the machine housing can stand on the floor with its rollers and can be pushed to another location by the user.

Moreover, the system can include electrically conductive shielding against electromagnetic radiation, which at least partly shields an inside region of the shielding against electromagnetic radiation from an outside region of the shielding.

In some embodiments, the electrically conductive shielding against electromagnetic radiation is shielding of the machine housing against electromagnetic radiation which at least partly shields an inside region of the machine housing against electromagnetic radiation from a region outside of the machine housing.

In some embodiments, the electrically conductive shielding of the machine housing only consists of an electrically conductive sheet metal or mesh behind a machine housing front made of plastic. In other embodiments, the electrically conductive shielding of the machine housing can shield the interior of the machine housing against outside electromagnetic radiation on a plurality of sides or on all sides.

In some embodiments, further or alternative electrically conductive shielding can be provided in the inside region or the outside region of the machine housing, as an alternative or in addition to the shielding of the machine housing.

In some embodiments, the electrically conductive shielding is a shielding housing. The shielding housing at least partly shields an inside region of the shielding housing against electromagnetic radiation from a region outside of the shielding housing.

The shielding housing may consist of or contain an electrically conductive material, such as an electrically conductive sheet metal.

The shielding housing may hold the components of the system that are susceptible to interference.

The shielding housing may be arranged in the inside region of the machine housing or in the outside region of the machine housing. By way of example, the shielding housing can be arranged in the interior of the electrically conductive shielding of the machine housing. Alternatively, the shielding housing can be fastened to the machine housing from the outside. In other embodiments, the shielding housing can be arranged in the outside region of the machine housing without being connected to the machine housing.

In some embodiments, the shielding housing may hold further components of the extracorporeal blood treatment machine susceptible to interference, for example an open-loop and closed-loop control apparatus and/or one or more central processing units (CPUs).

The system may further include a tubing receptacle apparatus, which is arranged in the region outside of the electrically conductive shielding against electromagnetic radiation. The tubing receptacle apparatus can include a tubing receptacle region which is configured for a reversible, holding reception of a tubing portion of a medical tubing line, in particular from the group of filtrate line, dialysate line, effluent line, for performing an extracorporeal blood treatment by means of the blood treatment machine for extracorporeal blood treatment.

In some embodiments, the tubing receptacle apparatus can be arranged on an outside surface of the machine housing.

In some embodiments, the tubing receptacle apparatus and/or the tubing receptacle region is configured for a reversible, holding reception of the tubing portion by pushing said tubing portion behind an undercut or behind a cross-sectional constriction of the tubing receptacle region by way of an elastic deformation of said tubing portion and/or for an interlocking hold by means of a housing door or a housing flap.

In some embodiments, the tubing receptacle apparatus is alternatively or cumulatively configured for an interlocking hold and/or a frictionally coupled hold of the tubing portion in the tubing receptacle region by means of a housing door or a housing flap. The housing door or the housing flap may be pivotably or rotatably mounted on the tubing receptacle apparatus by means of a hinge such that the housing door or the housing flap can be pivoted or rotated from a first, closed position, in which it covers the tubing receptacle region and prevents a tubing portion from falling out of or being removed from the tubing receptacle region, to a second, open position, in which the user can take the tubing portion from the tubing receptacle region or can insert the tubing portion into the tubing receptacle region. The tubing receptacle apparatus may have a lock for the housing door or the housing flap such that the housing door or the housing flap is not pivotable or rotatable in relation to the tubing receptacle apparatus in the first, closed position when the lock is closed. In particular, the lock for the housing door or the housing flap may be designed as a latching apparatus with a latching lock on the tubing receptacle apparatus.

By using light guides, the system facilitates the ability to arrange all components that are susceptible to interference by electromagnetic radiation, for example LEDs, phototransistors and the control circuit, on a printed circuit board outside of the tubing receptacle apparatus, in particular spatially separate from the tubing receptacle apparatus, within the shielding of the machine housing of the blood treatment machine and/or within the shielding housing.

Therefore, in some embodiments, the tubing receptacle apparatus includes or contains no LEDs, no sensors, no electrical components and/or electronic components, and no electrical lines or printed circuit boards or electrical cables. This is advantageous because this allows the interference susceptibility of the system in relation to electromagnetic radiation from the surroundings of the dialysis machine to be reduced, in particular the interference susceptibility of the phototransistors susceptible to interference since these are not contained in the tubing receptacle apparatus that is exposed to radiofrequency electromagnetic radiation from the surroundings in particular.

The use of light guides in the system moreover allows the electric safety of the patient to be further improved by reducing the patient leakage currents because possible clearances and leakage paths are entirely avoided in the region of the tubing receptacle apparatus if no electrical components whatsoever are arranged in the tubing receptacle apparatus.

In some embodiments, the tubing receptacle apparatus has or contains no or no separate electrically conductive shielding against electromagnetic radiation. This is advantageous because despite the constricted space in the tubing receptacle apparatus and constricted space at the front of the dialysis machine, a simpler and easier-to-repair structure can be ensured.

In some embodiments, the system includes an electronic measuring apparatus which has an electrical light source that, when activated, emits light into the first light guide, and has an optical sensor that receives light emerging from the second light guide, the electronic measuring apparatus being arranged in the inside region of the electrically conductive shielding.

In some embodiments, the electronic measuring apparatus includes a further optical sensor in addition to the optical sensor, said further optical sensor having a further phototransistor which is illuminated directly by the light source, when activated, by bypassing the first light guide and the second light guide.

The further phototransistor may be arranged and connected in the electronic measuring apparatus as a reference phototransistor, the reference phototransistor being illuminated directly by the light source during the operation thereof, by circumventing the first light guide and the second light guide.

In the electronic measuring apparatus, the optical sensor is spatially separated from the electrical light source by way of a light-opaque barrier in some embodiments, so that no light from the light source can strike the optical sensor directly by bypassing the first light guide and the second light guide.

In some embodiments, the electrical light source includes at least one light-emitting diode (LED) with a green colour spectrum and at least one LED with a red colour spectrum.

As a result of the use of light guides, all electrical and electronic components can be arranged into a region with more available space, in particular in the interior of the machine housing of the blood treatment machine. This yields the option of being able to simultaneously use a plurality of LEDs with the same green frequency spectrum such that the luminous intensity of the green light is advantageously increased.

In some embodiments, the electrical light source includes at least one second LED with a green colour spectrum, with, for simultaneous light generation, the first LED with a green colour spectrum and the second LED with a green colour spectrum being arranged and connected adjacently on a common printed circuit board. This achieves the advantage that ageing of the individual green LED is slowed down as the latter need not be operated under great wear-and-tear at its maximally specified electric current to provide the appropriate luminous intensity, but instead may be operated sparingly with a reduced electric current. Advantageously, the service life of the LEDs is increased, the maintenance and repair outlay is reduced and, ultimately, the overall costs are reduced as a result. This also increases the sensitivity of the blood leakage detector because the luminous intensity of two or more LEDs is greater than that of an individual LED despite a reduced operational electric current, in particular so that a computational blood loss of 0.35 ml/min or even less can be reliably detected over the entire range of the usual flow rates of the dialysate or of the filtrate or of the effluent.

In some embodiments, the system has a first light guide which is configured as an illumination source leading to the tubing receptacle region and which includes a first end portion and a second end portion, and has a second light guide which includes a third end portion and a fourth end portion, the second light guide being present in addition to the first light guide and being configured as light guide leading away from the tubing receptacle region for the purposes of transmitting light that emerges from the tubing portion.

According to the present disclosure, the medical system for optically detecting at least one blood constituent in a tubing line at a blood treatment machine for extracorporeal blood treatment can include an aperture region in the electrically conductive shielding against electromagnetic radiation, the first light guide and the second light guide being led from the outside region to the inside region of the shielding through the aperture region.

In this context, the overarching term “aperture region” describes all housing apertures through which the first light guide and the second light guide have to be led to be laid from the tubing receptacle apparatus in the outside region of the shielding to the measuring apparatus in the inside region of the electrically conductive shielding, to be precise independently of where the housing apertures are arranged and independently of the number of housing apertures through which the first light guide and the second light guide have to be led and independently of the number of shieldings provided and independently of how the various shieldings are arranged in space. As used herein, the term “aperture region” also includes embodiments in which provision is made for more than just one shielding, for example in which first shielding at least partly envelopes second shielding or in which first shielding and second shielding are present spatially separate from one another.

Therefore, the present disclosure can include specific embodiments in which the electrically conductive shielding against electromagnetic radiation is shielding or is only shielding of the machine housing against electromagnetic radiation. The present disclosure also encompasses other specific embodiments in which the electrically conductive shielding against electromagnetic radiation is a shielding housing. In particular, the present disclosure encompasses embodiments in which the shielding housing is arranged within or outside of the machine housing of the blood treatment machine. Moreover, the present disclosure encompasses specific embodiments in which the shielding housing is present in addition to the shielding of the machine housing of the blood treatment machine and is arranged within or outside of the shielding of the machine housing of the blood treatment machine.

In some embodiments, the first light guide and the second light guide are either led through the shielding through a common housing aperture or, alternatively, the first light guide is led through a first housing aperture and the second light guide is led through a second housing aperture, in each case through the shielding. What is disclosed if second shielding is present is that the first light guide and the second light guide are either led through the second shielding through a third common housing aperture or, alternatively, the first light guide is led through the third housing aperture and the second light guide is led through a fourth housing aperture, in each case through the second shielding.

In some embodiments, the system includes a first housing aperture which passes through a wall of the machine housing and/or the shielding of the machine housing of the blood treatment apparatus for extracorporeal blood treatment, the first housing aperture being configured to pass through the first light guide and the second light guide, and the at least one first light guide and the at least one second light guide being led into the inside region in the machine housing through the first housing aperture or being able to be led into the inside region of the machine housing through the first housing aperture for the assembly thereof.

In some embodiments, the system includes a first housing aperture and a second housing aperture which pass through a wall of the machine housing and/or the shielding of the machine housing of the blood treatment apparatus for extracorporeal blood treatment, the first housing aperture being configured to pass through the first light guide and the second housing aperture being configured to pass through the second light guide, and the first light guide being led into the inside region of the machine housing through the first housing aperture or being able to be led into the inside region of the machine housing through the first housing aperture for the assembly thereof and the second light guide being led into the inside region of the machine housing through the second housing aperture or being able to be led into the inside region of the machine housing through the second housing aperture for the assembly thereof.

In some embodiments, the system includes a third aperture as an alternative or in addition to the first housing aperture or as an alternative or in addition to the first housing aperture and the second housing aperture, said third aperture passing through a wall of the shielding housing and being configured to lead through the first light guide and the second light guide, the at least one first light guide and the at least one second light guide being led into the inside region of the shielding housing through the third housing aperture or being able to be led into the inside region of the shielding housing through the third housing aperture for the assembly thereof.

In some embodiments, the system includes a third aperture and a fourth aperture in addition or as an alternative to the first housing aperture or in addition or as an alternative to the first housing aperture and the second housing aperture, said third and fourth apertures passing through a wall of the shielding housing, the third housing aperture being configured to pass through the first light guide and the fourth housing aperture being configured to pass through the second light guide, and the first light guide being led into the inside region of the shielding housing through the third housing aperture or being able to be led into the inside region of the shielding housing through the third housing aperture for the assembly thereof and the second light guide being led into the inside region of the shielding housing through the fourth housing aperture or being able to be led into the inside region of the shielding housing through the fourth housing aperture for the assembly thereof.

In some embodiments, the first end portion of the first light guide in the tubing receptacle apparatus is aligned at the tubing receptacle portion so that when the tubing portion is inserted, the light rays emerging from the first end portion illuminate the tubing portion and the second end portion of the first light guide at the electronic measuring apparatus is aligned so that the light rays emerging from the light source enter the second end portion of the first light guide.

In some embodiments, the third end portion of the second light guide in the tubing receptacle apparatus is aligned at the tubing receptacle portion so that when the tubing portion is inserted, the light rays passing through the tubing portion enter the third end portion of the second light guide and the fourth end portion of the second light guide at the electronic measuring apparatus is aligned so that the light rays emerging from the fourth end portion strike the optical sensor.

In some embodiments, the first and the third end portion can be aligned in the tubing receptacle apparatus by means of, e.g., cylindrical channels or bores in the tubing receptacle apparatus. In specific embodiments, in turn, the tubing receptacle apparatus is produced by injection moulding and already has the cylindrical channels, and so the end portions are inserted into the cylindrical channels for the assembly thereof. The first and the third end portion can be aligned by means of two cylindrical sleeves which are each fastened to the printed circuit board, the end portions being inserted into the cylindrical sleeves for the assembly thereof.

In some embodiments, the first light guide and the second light guide each are flexible light-guide cables or each have flexible light-guide cables. This provides the advantage that the electronic measuring apparatus can be arranged in the inside region of the machine housing independently of the distance from the tubing receptacle apparatus, in particular at locations that are advantageously protected from electromagnetic radiation generated in the interior of the machine housing and at locations that are accessible for ease of assembly and ease of maintenance, both during assembly in the factory and for a service technician at a clinic.

In special embodiments, the flexible light-guide cables each have or consist of a plurality or multiplicity of light-guiding glass optical fibres or plastic optical fibres.

In some embodiments, the first light guide and the second light guide are each rigid light-guiding optical components, in particular made of plastic, in particular made of polycarbonate (PC) or polymethylmethacrylate (PMMA) or acrylonitrile butadiene styrene (ABS), which may be produced by means of injection moulding for example.

A blood treatment machine for extracorporeal blood treatment is described. In some embodiments, the blood treatment machine includes a medical system for optically detecting at least one blood constituent in the tubing portion of the medical tubing line, the medical tubing line with the tubing portion being part of a disposable tubing set, additionally includes a blood pump which is configured to convey blood in at least one blood tubing line which is part of the disposable tubing set and which should be connected on for the extracorporeal blood treatment. The blood treatment machine can include a machine-side venous clamp for clamping the venous line or return line which is part of the disposable tubing set, and it includes at least one further pump which is configured to pump a medical fluid, intended to be discarded, in the medical tubing line and the tubing portion. The blood treatment machine can include a control and computing unit with a memory in which a computer program is stored, the latter, when executed, controlling the electronic measuring apparatus and evaluating the measurement data of the optical sensor and establishing an inadmissibly high proportion of blood or a blood constituent in the tubing portion when a limit value is exceeded and the control and computing unit being programmed to output an alarm and to stop at least the blood pump for the purposes of establishing a safe state and to close the machine-side venous clamp if an inadmissibly high proportion of blood or a blood constituent is established.

In particular, the medical tubing line can be a tubing line from the group of effluent line, dialysate line, filtrate line.

The pump for pumping the medical fluid, to be discarded, in the medical tubing line and the tubing portion can be, in particular, a pump from the group of filtrate pump, effluent pump, dialysate pump.

The tubing portion can be configured for functional coupling in the tubing receptacle by way of at least one feature of the following group: external diameter, length, deformability, translucence, reflectance, wall strength.

The blood tubing line to be connected for the extracorporeal blood treatment may be selected from the group of: withdrawal line, arterial line, return line, venous line.

Since, the system for optically detecting at least one blood constituent in a tubing line at a blood treatment machine for extracorporeal blood treatment is suitable for enabling a computer program with an evaluation algorithm for blood leakage detection that has proven its worth in the prior art, in particular by the multiFiltratePRO dialysis machine of the applicant, to run in the memory of the measuring apparatus, the system does not require costly software modifications to attain the advantages rendered by aspects of the present disclosure.

The computer program in the memory of the control and computing unit may additionally be programmed to recognize a fluid-free tubing portion in the tubing receptacle region prior to the start of an extracorporeal blood treatment and to output an alarm if no fluid-free tubing portion is recognized in the tubing receptacle region.

In some embodiments, blood treatment machine can be selected from the group of: haemodialysis machine, haemofiltration machine, haemodiafiltration machine, acute dialysis machine, CRRT machine.

### BRIEF DESCRIPTION OF FIGURES

FIG. 1 shows a schematic illustration of an extracorporeal blood circuit.

FIG. 2 shows a view of an exemplary blood treatment machine for extracorporeal blood treatment.

FIG. 3 shows a schematic illustration of an exemplary embodiment of a medical system for optically detecting at least one blood constituent in a tubing line.

FIG. 4 shows a schematic illustration of an exemplary embodiment of a disposable tubing set for the extracorporeal blood treatment.

FIG. 5 shows a view of an example tubing receptacle apparatus.

An exemplary embodiment of the medical system for optically detecting at least one blood constituent in a tubing line at a blood treatment machine and a blood treatment machine is described in more detail below, with reference being made to the drawings. Further details and advantages of the present disclosure are described in more detail on the basis of the exemplary embodiment described in the drawings. The meaning of a respective reference sign in the drawings is the same throughout all the drawings.

### DETAILED DESCRIPTION

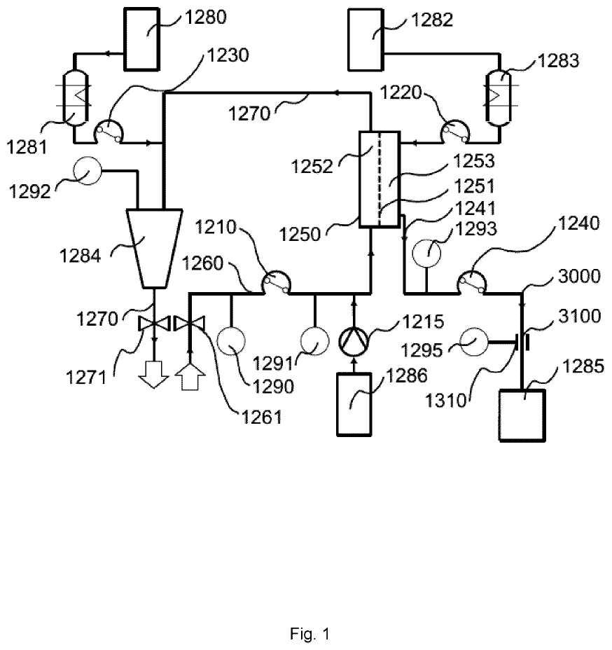

FIG. 1 shows a schematic illustration of an extracorporeal blood circuit as part of a system according to the present disclosure. The extracorporeal blood circuit comprises a withdrawal line or arterial blood line 1260 with a blood pump 1210, by means of which the blood is aspirated from a vascular access of a patient and led to the blood chamber of a dialyser or blood filter 1250. From the blood chamber 1252, the blood is led back into the vascular access of the patient by means of a return line or venous line 1270 with an air bubble separator or a venous chamber 1284. The patient-side ends of the withdrawal line or arterial blood line 1260 and of the return line or venous line 1270 have a removal clamp or arterial clamp 1261 and a return clamp or venous clamp 1271, respectively, by means of which the flow can be interrupted by clamping the tubing line by way of machine-side actuators.

The dialyser or blood filter 1250 is divided in its interior into the blood chamber and a dialysate chamber or filtrate chamber by way of a semipermeable membrane 1251. In the case of haemodialysis, a dialysis fluid is fed to the dialysate chamber by means of a dialysis fluid pump 1220 in a dialysis fluid line, the dialysis fluid being provided in a dialysis fluid container 1282. On its way to the dialysate chamber, the dialysis fluid may be heated in a dialysis fluid heating bag 1283. During haemodialysis, substances to be separated from the blood diffuse from the blood into the dialysate chamber through the semipermeable membrane. The dialysate which is loaded with the substances to be separated and which is to be discarded is carried away to a dialysate container 1285 by means of a dialysate pump 1240 in a medical tubing line 3000. In the case of purely convective haemofiltration, filtrate passes from the blood chamber to the filtrate chamber 1253 through the semipermeable membrane. In the case of haemodiafiltration, which is both a diffusive and convective method, filtrate likewise passes from the blood chamber to the filtrate chamber 1253 through the semipermeable membrane and is pumped into a filtrate container 1285 by means of the filtrate pump 1240 in the medical tubing line 3000. The fluid, to be discarded, in the medical tubing line 3000, regardless of whether it is referred to as dialysate or filtrate or simply as effluent, in this case flows through a tubing portion 3100, on which an optical blood leakage detector 1295 is arranged, before it is collected for disposal in the dialysate container or filtrate container or effluent container 1285. In the present disclosure, the terms dialysate, filtrate, effluent are used synonymously for the fluid to be discarded since the blood treatment methods used to obtain the fluid to be discarded is unimportant in the case of the present disclosure. The optical blood leakage detector 1295 recognizes blood or blood constituents with a certain sensitivity in the dialysate, filtrate or effluent while this flows through the tubing portion 3100 at a certain flow rate. In some embodiments, the extracorporeal blood circuit may have various pressure measuring points, specifically a pressure measuring point 1290 upstream of the blood pump, a pressure measuring point 1291 downstream of the blood pump, upstream 1291 and a pressure measuring point 1293 at the return line or venous line 1270, in particular at the air bubble separator or the venous chamber 1284. In some embodiments, a substitution fluid line with a substitution fluid container 1280, a substitution fluid pump 1230 and a substitution fluid heating bag 1281 may additionally be provided. In FIG. 1, the substitution fluid line is shown at the return line or venous line 1270 in exemplary fashion. Alternatively, or additionally, a corresponding arrangement of a further substitution fluid line with a further substitution fluid container, a further substitution fluid pump and a further substitution fluid heating bag may also be provided at the withdrawal line or arterial line 1260.

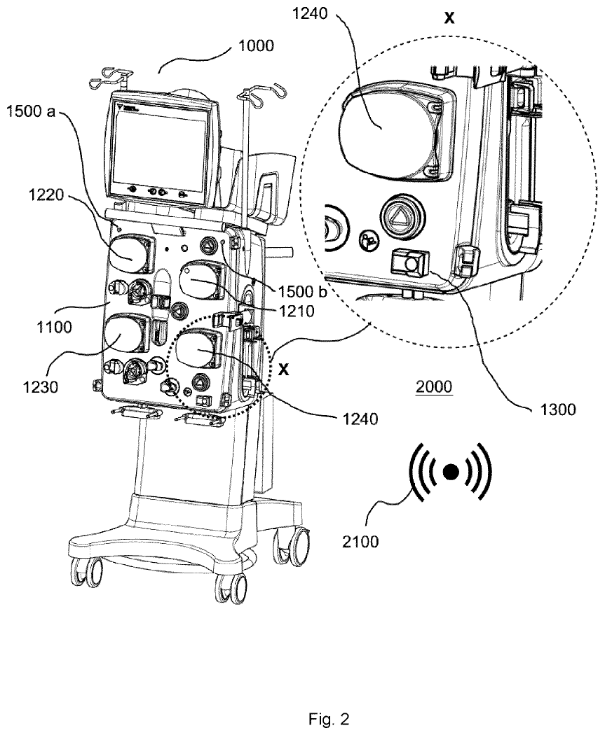

FIG. 2 shows a schematic perspective view of a blood treatment machine 1000 for extracorporeal blood treatment and a medical system for optically detecting at least one blood constituent in a tubing line at the blood treatment machine 1000 for extracorporeal blood treatment. A component of a blood leakage detector system, specifically a tubing receptacle apparatus 1300, is depicted in an enlarged detail represented by a dashed circular line in FIG. 1. The tubing receptacle apparatus 1300 is configured such that a tubing portion 3100 of a medical tubing line 3000 can be reversibly received and held in a tubing receptacle region 1310 arranged in the tubing receptacle apparatus 1300. The tubing portion 3100 and the medical tubing line 3000 are part of a disposable tubing set 4000, which is functionally connected by the user by hand at the front of the blood treatment machine 1000 for extracorporeal blood treatment for the purposes of performing an extracorporeal blood treatment session.

In specific embodiments of a disposable tubing set 4000, the disposable tubing set 4000 may be pre-equipped in or on an organiser or tray to simplify the functional coupling. In such embodiments, the functional coupling of the disposable tubing set 4000, which is also referred to as the set-up procedure by the user, starts by manually hooking the organiser or tray to two hooks or pins 1500a and 1500b at the front of the blood treatment machine 1000. As a result, all tubing set components to be functionally connected hang, in user-friendly fashion, in front of the associated machine-side actuators and sensors. By way of example, the medical tube line 3000 is arranged by means of the tray or organiser such that the tubing portion 3100, in user-friendly fashion, hangs directly in front of the tubing receptacle apparatus 1300. This avoids errors during the set-up as a result of mistakenly mixing up tubing portions and a particularly quick and user-friendly set-up procedure is rendered possible, this being particularly important in intensive-care medicine or emergency medicine in intensive care units.

At the front of the blood treatment machine 1000, FIG. 2 schematically shows a dialysis fluid pump 1220 which is configured for functionally connecting a dialysis fluid pump tubing segment 1221, a substitution fluid pump 1230 which is configured for functionally connecting a substitution fluid pump tubing segment 1231, a filtrate pump or effluent pump or dialysate pump 1240, which is configured for functionally connecting a filtrate pump tubing segment 1241 and a blood pump 1210 which is configured for functionally connecting a blood line pump tubing segment 1262. The aforementioned pump tubing segments are components of the disposable tubing set 4000.

Further pumps may be added in specific embodiments, for example a heparin pump for performing heparin anticoagulation and/or for performing regional anticoagulation, a citrate pump for pumping a citrate-containing medical solution and a calcium pump for pumping a calcium-containing medical solution. In such embodiments, the disposable tubing set 4000 additionally comprises at least one heparin line, which opens into the arterial blood line or withdrawal line, and a citrate line, which likewise opens into the arterial blood line or withdrawal line, and a calcium line, which opens into the venous blood line or return line.

Moreover, the pictogram with the reference sign 2100 in FIG. 2 schematically indicates electromagnetic radiation from the surroundings in a region 2000 outside of the machine housing 1100 of the blood treatment machine 1000.

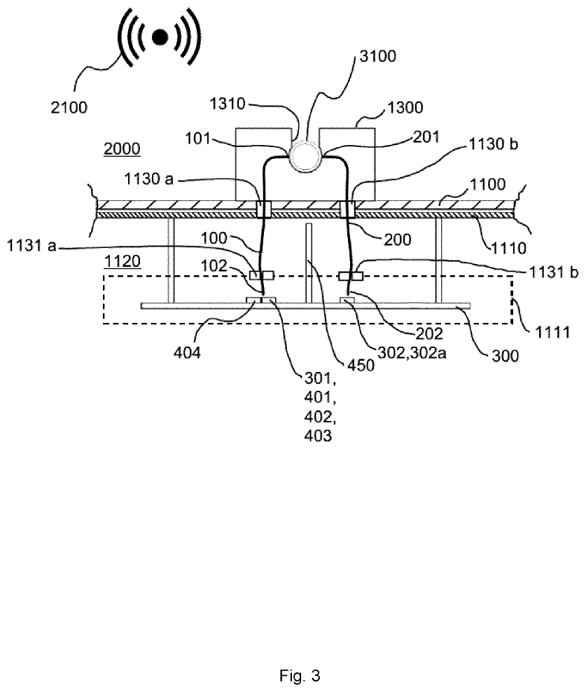

FIG. 3 schematically shows the structure of a medical system for optically detecting at least one blood constituent in a tubing line at a blood treatment machine for extracorporeal blood treatment 1000.

Moreover, the pictogram with the reference sign 2100 in FIG. 3 schematically indicates electromagnetic radiation from the surroundings in a region 2000 outside of the machine housing 1100 of the blood treatment machine 1000. The system comprises a blood treatment machine for extracorporeal blood treatment 1000 with a machine housing 1100 with at least piecewise shielding 1110 of the machine housing 1100 against electromagnetic radiation, the shielding 1110 at least partly shielding an inside region of the machine housing 1100 against electromagnetic radiation 2100 from a region 2000 outside of the machine housing 1100.

In specific embodiments, the shielding 1110 of the machine housing 1100 only consists of a sheet metal behind a machine housing front made of plastic. In some embodiments, the shielding 1110 of the machine housing 1100 can shield the machine housing against outside electromagnetic radiation on a plurality of sides or on all sides.

The system further comprises a tubing receptacle apparatus 1300 on an outer surface of the machine housing 1100 in the region outside of the shielding 1110, the tubing receptacle apparatus comprising a tubing receptacle region 1310 which is configured for a reversible, holding reception of a tubing portion 3100 of a medical tubing line 3000 from the group of filtrate line, dialysate line, effluent line, for performing an extracorporeal blood treatment by means of the blood treatment machine for extracorporeal blood treatment 1000.

In the embodiment shown in FIG. 3, shielding is provided in the inside region 1120 of the machine housing 1100 in addition to the shielding 1110 of the machine housing. In some embodiments, the shielding in the inside region 1120 of the machine housing 1100 is a shielding housing 1111 made of sheet metal. In FIG. 3, the shielding housing 1111 is indicated by a dashed line. The use of light guides allows the measurement electronics to be arranged within the shielding housing 1111 at a spatial distance from the tubing receptacle apparatus 1300. The use of flexible light-guide cables in particular promotes simple laying of the light guides from the tubing receptacle apparatus 1300 to the electronic measuring apparatus 300, even over relatively long distances within the machine housing.

The system further has a first light guide 100 which is configured as an illumination source leading to the tubing receptacle region 1310 and which comprises a first end portion 101 and a second end portion 102, and has a second light guide 200 which comprises a third end portion 201 and a fourth end portion 202, the second light guide being present in addition to the first light guide 100 and being configured as light guide leading away from the tubing receptacle region for the purposes of transmitting light that emerges from the tubing portion 3100 when the system is used as intended.

The machine housing 1100 has at least one housing aperture 1130, 1130a, 1130b, which passes through the shielding of the machine housing 1100 of the blood treatment apparatus for extracorporeal blood treatment 1000, wherein, in the operationally ready state, the first light guide 100 and the second light guide 200 are led into the inside region 1120 of the machine housing 1100 through the at least one housing aperture 1130, 1130a, 1130b or are able to be led into the inside region 1120 of the machine housing 1100 through the at least one housing aperture 1130, 1130a, 1130b for the assembly thereof. The at least one housing aperture is a passage opening. It may preferably have a round or polygonal cross section and be produced by drilling or punching. The aperture cross section of the at least one housing aperture is larger than the cross section of the light guide to be led through. Two separate housing apertures 1130a, 1130b are present in the exemplary embodiment shown in FIG. 3, and so each one of the two light guides is led through its own housing aperture. However, both light guides may also be led through a single housing aperture 1130 in specific embodiments.

The system has an electronic measuring apparatus 300, which is arranged in the inside region 1120 of the machine housing 1100 and comprises an electrical light source 301 for emitting light into the first light guide 100 and an optical sensor 302 for receiving light emerging from the second light guide 200.

The tubing receptacle apparatus comprises or contains no LEDs, no sensors, no electrical components and/or electronic components, and no electrical lines or printed circuit boards or electrical cables. This is advantageous because this allows the interference susceptibility of the system in relation to electromagnetic radiation from the surroundings of the dialysis machine to be reduced, in particular the interference susceptibility of the phototransistors susceptible to interference since these components are not contained in the tubing receptacle apparatus that is exposed to radiofrequency electromagnetic radiation from the surroundings in particular, but instead are contained within the shielding 1110 of the machine housing 1100.

Moreover, the tubing receptacle apparatus 1300 has or contains no shielding against electromagnetic radiation. This is advantageous because despite the constricted space in the tubing receptacle apparatus and constricted space at the front of the dialysis machine, a simple and easy-to-repair structure can thereby be ensured.

As shown in FIG. 3, the first end portion 101 of the first light guide 100 in the assembled state in the tubing receptacle apparatus 1300 is aligned at the tubing receptacle portion 1310 so that during the intended operation of the system with an inserted tubing portion 3100, the light rays emerging from the first end portion 101 illuminate the tubing portion 3100 and that the second end portion 102 of the first light guide 100 in the assembled state at the electronic measuring apparatus 400 is aligned so that during the intended operation of the system, the light rays emerging from the light source 301 enter the second end portion 102 of the first light guide 100.

As shown further in FIG. 3, the third end 201 of the second light guide 200 in the assembled state in the tubing receptacle apparatus 1300 is aligned at the tubing receptacle portion 1310 so that during the intended operation of the system with an inserted tubing portion 3100, the light rays passing through the tubing portion 3100 enter the third end 201 of the second light guide 200 and that the fourth end portion 202 of the second light guide 200 in the assembled state at the electronic measuring apparatus 300 is aligned so that during the intended operation of the system, the light rays emerging from the fourth end portion 202 strike the optical sensor 302, the optical sensor 302 having a phototransistor 302a.

In the exemplary embodiment shown, the first light guide 100 and the second light guide 200 each are flexible light-guide cables or each have flexible light-guide cables. This provides the advantage that the electronic measuring apparatus 300 can be arranged in the inside region 1120 of the machine housing 1100 independently of the distance from the tubing receptacle apparatus 1300, in particular at locations that are advantageously protected from electromagnetic radiation generated in the interior or outside of the machine housing 1100 and at locations that are accessible for ease of assembly and ease of maintenance, both during assembly in the factory and for a service technician at a clinic.

In the exemplary embodiment shown, the flexible light-guide cables each have or consist of a plurality of light-guiding glass optical fibres or plastic optical fibres.

In the exemplary embodiment shown in FIG. 3, the electronic measuring apparatus 300 has two LEDs 401, 402 with a green colour spectrum as an electrical light source 301, the first LED 401 with a green colour spectrum and the second LED 402 with a green colour spectrum being arranged and connected on a printed circuit board 400 for the simultaneous generation of light. This achieves the advantage that ageing of the green LED is slowed down as the latter need not be operated under great wear-and-tear at its maximally specified electric current to provide the desired luminous intensity, but instead may be operated sparingly with a reduced electric current because the desired luminous intensity overall is provided by two LEDs. Advantageously, the service life of the green LEDs is increased, the maintenance and repair outlay is reduced and, ultimately, the overall costs are reduced as a result. This also increases the sensitivity of the blood leakage detector because the luminous intensity of two or more LEDs is greater than that of an individual LED despite a reduced operational electric current, in particular so that a computational blood loss of 0.35 ml/min or even less can be reliably detected over the entire range of usual flow rates of dialysate or of filtrate or of effluent.

A reference phototransistor 404 which, for the purposes of carrying out a reference measurement, is illuminated by the light source 301 during the operation of the latter is arranged and connected in the electronic measuring apparatus 300.

In the electronic measuring apparatus 300, the phototransistor 302a is spatially separated from the electrical light source 301 by way of a light-opaque barrier 450, so that no light from the light source 301 can strike the phototransistor 302a directly by bypassing the first light guide 100 and the second light guide 200.

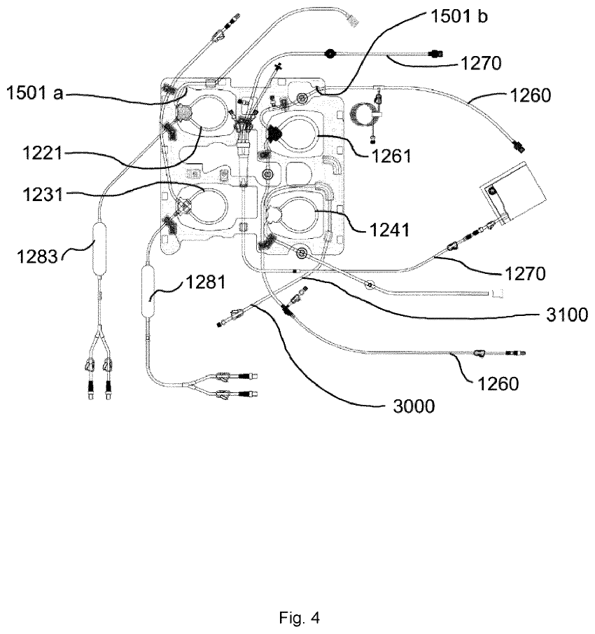

FIG. 4 shows an exemplary embodiment of a disposable tubing set 4000, which is prepared on an organiser or tray in user-friendly fashion and so as to prevent mix-ups. By way of example, the organiser or the tray consists of a thermoformed plastic film and has cut-outs or perforations in the region of the pumps, the cut-outs forming two ears 1501a and 1501b at the same time and being configured to suspend and align the disposable tubing set 4000 at the two hooks or pins 1500a and 1500b at the front of the blood treatment machine 1000, as shown in FIG. 2.

In the exemplary embodiment shown, the medical tubing line 3000 with the tubing portion 3100 is part of the disposable tubing set 4000.

In the embodiment shown, the medical tubing line 3000 is affixed at a specified position on the organiser or tray by means of at least one clip, and so the tubing portion 3100 hangs in user-friendly fashion and so as to prevent mix-ups in front of the tubing receptacle region of the tubing receptacle apparatus 1300 (see FIG. 2) when the organiser or the tray is suspended from the two hooks or pins 150a and 1500b at the front of the blood treatment machine 1000.

In the embodiment shown in FIG. 4, the disposable tubing set 4000 has a blood tubing line or withdrawal line or arterial line 1260 with a blood line pump tubing segment 1262 and a return line or venous line 1270 with an air bubble separator or a venous chamber 1284. Moreover, a dialysis fluid pump tubing segment 1221, a substitution fluid pump tubing segment 1231 and a filtrate pump tubing segment 1241 are shown. The pump tubing segments 1262, 1221, 1231 and 1241 are positioned and affixed on the organiser or tray in such a way that these are aligned and assigned in front of the roller pumps in user-friendly fashion and so as to prevent mix-ups when the organiser or the tray is suspended from the two hooks or pins 1500a and 1500b at the front of the blood treatment machine 1000 (see FIG. 2) and said pump tubing segments can each be functionally coupled to the respectively intended roller pump by the user in a manner not allowing mix-ups or can be inserted into said roller pump for functional coupling purposes.

The dialysis fluid line has a dialysis fluid heating bag 1283 and the substitution fluid line has a substitution fluid heating bag 1281.

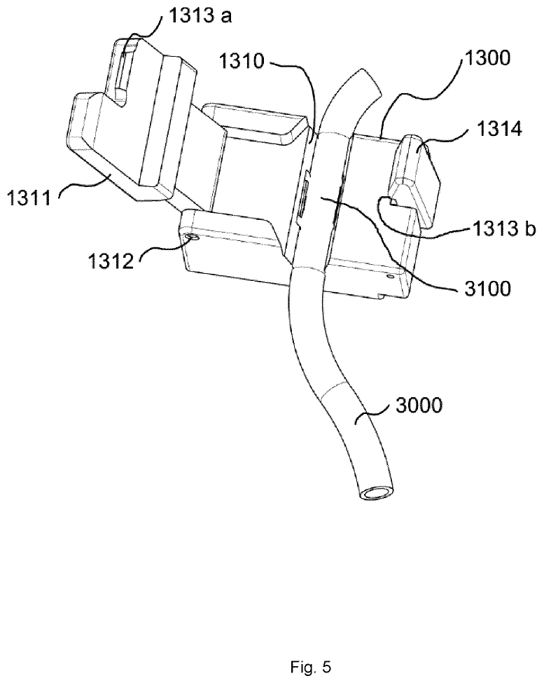

FIG. 5 shows a perspective view of an exemplary constructive embodiment of a tubing receptacle apparatus 1300 with a tubing receptacle region 1310. The tubing receptacle apparatus 1300 has a housing door 1311 which is movably mounted on a hinge 1312 and which covers the tubing receptacle region 1310 and prevents a tubing portion 3100 from falling out of or being removed from the tubing receptacle region 1310 in a first, closed position and which gives access to the tubing receptacle region 1310 in a second, open position (shown in FIG. 5). The housing door 1311 has a latching apparatus 1313a, 1313b, which is configured for reversible latching with a latching lock 1314 when the housing door 1311 is in the first, closed position. For the exemplary embodiment, the perspective view of FIG. 5 shows an only indicated medical tubing line 3000 with a tubing portion 3100 in a functionally coupled state, which facilitates the coupling of light into the tubing portion 3100 and the output coupling and transmission of transmission light from the tubing portion 3100 when the system is in intended operation. The detachable arrangement of the light guides in the interior of the tubing receptacle apparatus 1300 and the further guidance of the light guides in the outside region of the tubing receptacle apparatus 1300 can be seen in FIG. 3. The tubing receptacle apparatus 1300 is moreover configured to be fastened to the machine front of the machine housing 1100, in particular by screwing. A further advantage of the tubing receptacle apparatus 1300 is that, in the case of damage, for example a broken housing door, it can easily be replaced by a replacement housing of the same design without having to replace the light guides or disassemble the entire system.

A number of embodiments have been described. Nevertheless, it will be understood that various modifications can be made without departing from the spirit and scope of the present disclosure. Accordingly, other embodiments are within the scope of the claims.

List of reference numerals

| | |
|---|---|
| 100 | First light guide |
| 101 | First end portion |
| 102 | Second end portion |
| 200 | Second light guide |
| 201 | Third end portion |
| 202 | Fourth end portion |
| 300 | Electronic measuring apparatus |
| 301 | Electrical light source |
| 302 | Optical sensor |
| 302a | Phototransistor |
| 400 | Printed circuit board |
| 401 | First LED with a green colour spectrum |
| 402 | Second LED with a green colour spectrum |
| 303 | LED with a red colour spectrum |
| 404 | Phototransistor |
| 450 | Light-opaque barrier |
| 1000 | Blood treatment machine for extracorporeal blood treatment |
| 1100 | Machine housing |
| 1110 | Shielding (of the machine housing) |
| 1111 | Shielding box, shielding housing |
| 1120 | Inside region of the machine housing |
| 1130 | Housing aperture in the machine housing |
| 1130a, | First and second housing aperture in the machine housing |
| 1130b | |
| 1131 | Housing aperture in the shielding housing |
| 1131a, | First and second housing aperture in the shielding housing |
| 1131b | |
| 1210 | Blood pump |
| 1215 | Heparin pump |
| 1220 | Dialysis fluid pump |
| 1230 | Substitution fluid pump |
| 1231 | Substitution fluid pump tubing segment |
| 1240 | Filtrate pump, effluent pump, dialysate pump |
| 1241 | Filtrate pump tubing segment |
| 1250 | Dialyser, blood filter |
| 1251 | Semipermeable membrane |
| 1252 | Blood chamber |
| 1253 | Dialysate chamber, filtrate chamber |
| 1260 | Blood tubing line, withdrawal line, arterial line |
| 1261 | Withdrawal clamp or arterial clamp |
| 1262 | Blood line pump tubing segment |
| 1270 | Return line, venous line |
| 1271 | Return clamp, venous clamp |
| 1280 | Substitution fluid container |
| 1281 | Substitution fluid heating bag |
| 1282 | Dialysis fluid container |
| 1283 | Dialysis fluid heating bag |
| 1284 | Air bubble separator, venous chamber |
| 1285 | Filtrate container, effluent container, dialysate container |
| 1286 | Heparin container |
| 1290 | Pressure measuring equipment upstream of the blood pump |
| 1291 | Pressure measuring equipment downstream of the blood pump |
| 1292 | Pressure measuring equipment for return pressure, venous |
| | pressure |
| 1295 | Optical blood leakage detector |
| 1300 | Tubing receptacle apparatus |
| 1310 | Tubing receptacle region |
| 1311 | Housing door |
| 1312 | Hinge |
| 1313a, | Latching apparatus |
| 1313b | |
| 1500a, | Hook, pin |
| 1500b | |
| 2000 | Region outside of the machine housing |
| 2100 | Electromagnetic radiation of the surroundings |
| 3000 | Medical tube line |
| 3100 | Tubing portion |
| 4000 | Disposable tubing set |

1-20. (canceled)

21. A medical system for optically detecting at least one blood constituent in a tubing line at a blood treatment machine for extracorporeal blood treatment, the medical system comprising:
the blood treatment machine for extracorporeal blood treatment, the blood treatment machine comprising:
a machine housing,
electrically conductive shielding against electromagnetic radiation that at least partly shields an inside region of the electrically conductive shielding from electromagnetic radiation from an outside region of the electrically conductive shielding,
a tubing receptacle apparatus arranged on the outside region of the electrically conductive shielding, the tubing receptacle apparatus comprising a tubing receptacle region configured for a reversible, holding reception of a tubing portion of a medical tubing line,
a first light guide configured as an illumination source leading to the tubing receptacle region, the first guide light comprising a first end portion and a second end portion,
a second light guide comprising a third end portion and a fourth end portion, the second light guide being present in addition to the first light guide and being configured as light guide leading away from the tubing receptacle region for the purpose of transmitting light that emerges from the tubing portion, and
an aperture region in the electrically conductive shielding against electromagnetic radiation, the first light guide and the second light guide being led from the outside region to the inside region of the electrically conductive shielding through the aperture region;
an electronic measuring apparatus arranged in the inside region of the electrically conductive shielding, the electronic measuring apparatus comprising:
an electrical light source that emits light into the first light guide, and
an optical sensor that receives light emerging from the second light guide.

22. The medical system according to claim 21, wherein the electrically conductive shielding against electromagnetic radiation is shielding of the machine housing against electromagnetic radiation.

23. The medical system according to claim 21, wherein the electrically conductive shielding against electromagnetic radiation is a shielding housing.

24. The medical system according to claim 23, wherein the shielding housing is arranged within or outside of the machine housing.

25. The medical system according to claim 24, wherein the shielding housing is present in addition to a shielding of the machine housing and is arranged within or outside of the shielding of the machine housing.

26. The medical system according to claim 21, wherein the tubing receptacle apparatus comprises or contains no LEDs, no sensors, no electrical components and/or electronic components, and no electrical lines or printed circuit boards or electrical cables.

27. The medical system according to claim 21, wherein the tubing receptacle apparatus comprises or contains no electrically conductive shielding against electromagnetic radiation.

28. The medical system according to claim 21, wherein the first light guide and the second light guide each are flexible light-guide cables and/or each comprise flexible light-guide cables.

29. The medical system according to claim 28, wherein the flexible light-guide cables each comprise a plurality of light-guiding glass optical fibres or plastic optical fibres.

30. The medical system according to claim 21, wherein the electrical light source comprises a first LED with a green colour spectrum and at least one LED with a red colour spectrum.

31. The medical system according to claim 30, wherein the electrical light source comprises a second LED with the green colour spectrum and, for simultaneous light generation, the first LED with the green colour spectrum and the second LED with the green colour spectrum are arranged and connected adjacently on a common printed circuit board.

32. The medical system according to claim 21, wherein the optical sensor comprises a phototransistor.

33. The medical system according to claim 32, wherein the electronic measuring apparatus comprises a second optical sensor with a second phototransistor, the second phototransistor being illuminated directly by the electrical light source by bypassing the first light guide and the second light guide.

34. The medical system according to claim 21, wherein, in the electronic measuring apparatus, the optical sensor is spatially separated from the electrical light source by way of a light-opaque barrier so that no light from the electrical light source can strike the optical sensor directly by bypassing the first light guide and the second light guide.

35. The medical system according to claim 21, wherein:
the first end portion of the first light guide in the tubing receptacle apparatus is aligned at the tubing receptacle region so that when the tubing portion is inserted, light rays emerging from the first end portion illuminate the tubing portion, and
the second end portion of the first light guide at the electronic measuring apparatus is aligned so that light rays emerging from the electrical light source enter the second end portion of the first light guide.

36. The medical system according to claim 21, wherein:
the third end portion of the second light guide in the tubing receptacle apparatus is aligned at the tubing receptacle region so that when the tubing portion is inserted, light rays passing through the tubing portion enter the third end portion of the second light guide, and
the fourth end portion of the second light guide at the electronic measuring apparatus is aligned so that light rays emerging from the fourth end portion strike the optical sensor.

37. The medical system according to claim 21, wherein the tubing receptacle apparatus and/or the tubing receptacle region is configured for the reversible, holding reception of the tubing portion by pushing the tubing portion behind an undercut or behind a cross-sectional constriction of the tubing receptacle region by way of an elastic deformation of the tubing portion and/or for an interlocking hold by means of a housing door or a housing flap.

38. The medical system according to claim 21, wherein the medical system is configured for optically detecting at least one blood constituent in the tubing portion of the medical tubing line, the medical tubing line with the tubing portion being part of a disposable tubing set, wherein the medical system comprises a blood treatment machine comprising:
a blood pump configured to convey blood in at least one blood tubing line of the disposable tubing set and that is connected for the extracorporeal blood treatment,
a machine-side venous clamp configured for clamping a venous line or return line of the disposable tubing set,
at least one further pump configured to pump a medical fluid in the medical tubing line and the tubing portion,
a control and computing unit with a memory configured to store a computer program that, when executed, controls the electronic measuring apparatus and evaluates measurement data of the optical sensor and establishes an inadmissibly high proportion of blood or a blood constituent in the tubing portion when a limit is exceeded,
the control and computing unit being programmed to output an alarm and to stop at least the blood pump for the purpose of establishing a safe state and to close the machine-side venous clamp if an inadmissibly high proportion of blood or blood constituent is established.

39. The medical system according to claim 38, wherein the computer program is further programmed to recognize a fluid-free tubing portion in the tubing receptacle region prior to the start of an extracorporeal blood treatment and to output the alarm if no fluid-free tubing portion is recognized in the tubing receptacle region.

40. The medical system according to claim 38, wherein the blood treatment machine is selected from a group consisting of a haemodialysis machine, a haemofiltration machine, a haemodiafiltration machine, an acute dialysis machine, and a CRRT machine.
